(19) United States
(12) Patent Application Publication
(10) Pub. No.: US 2017/0367828 A1
(43) Pub. Date: Dec. 28, 2017

(54) Interpositional Joint Implant

(71) Applicant: ConforMIS, Inc., Billerica, MA (US)

(72) Inventors: Daniel Steines, Lexington, MA (US); Philipp Lang, Lexington, MA (US); Wolfgang Fitz, Sherborn, MA (US)

(21) Appl. No.: 15/589,222

(22) Filed: May 8, 2017

Related U.S. Application Data
(63) Continuation of application No. 11/688,340, filed on Mar. 20, 2007, now abandoned.
Application No. 11/688,340 is a continuation-in-part of application No. 10/997,407, filed on Nov. 24, 2004, now Pat. No. 8,882,847.
Application No. 10/997,407 is a continuation-in-part of application No. 10/752,438, filed on Jan. 5, 2004, now Pat. No. 8,545,569.
Application No. 10/752,438 is a continuation-in-part of application No. 10/724,010, filed on Nov. 25, 2003, now Pat. No. 7,618,451.
Application No. 10/724,010 is a continuation-in-part of application No. 10/305,652, filed on Nov. 27, 2002, now Pat. No. 7,468,075.
Application No. 10/305,652 is a continuation-in-part of application No. 10/160,667, filed on May 28, 2002, now abandoned.
Application No. 11/688,340 is a continuation-in-part of application No. 10/681,750, filed on Oct. 7, 2003, now abandoned.
Application No. 11/688,340 is a continuation-in-part of application No. 10/681,749, filed on Oct. 7, 2003, now Pat. No. 7,799,077.
Application No. 10/681,749 is a continuation-in-part of application No. 11/671,745, filed on Feb. 6, 2007, now Pat. No. 8,066,708.
(60) Provisional application No. 60/784,255, filed on Mar. 21, 2006.
(60) Provisional application No. 60/293,488, filed on May 25, 2001.
(60) Provisional application No. 60/363,527, filed on Mar. 12, 2002.
(60) Provisional application No. 60/380,695, filed on May 14, 2002.
(60) Provisional application No. 60/380,692, filed on May 14, 2002.
(60) Provisional application No. 60/467,686, filed on May 2, 2003.
(60) Provisional application No. 60/416,601, filed on Oct. 7, 2002.
(60) Provisional application No. 60/765,592, filed on Feb. 6, 2006.
(60) Provisional application No. 60/785,168, filed on Mar. 23, 2006.
(60) Provisional application No. 60/788,339, filed on Mar. 31, 2006.

(51) Int. Cl. A61F 2/30; A61B 5/107; A61F 2/38; A61B 5/103; A61F 2/46; A61B 5/00

(52) U.S. Cl. CPC A61F 2/30942; A61B 5/107; A61F 2/3872; A61F 2/30756; A61F 2310/00365; A61F 2310/00293; A61F 2310/00179; A61F 2310/00011; A61F 2250/0036; A61F 2230/0028; A61F 2230/0015; A61F 2230/0008; A61F 2210/0014; A61F 2210/0004; A61F 2002/4635; A61F 2002/30962; A61F 2002/30957; A61F 2002/30952; A61F 2002/30948; A61F 2002/30884; A61F 2002/30878; A61F 2002/30324; A61F 2002/30163; A61F 2002/30133; A61F 2002/30125; A61F 2002/30092; A61F 2002/3007; A61F 2002/30062; A61F 2/30767; A61B 5/4528; A61B 5/4514; A61B 5/1077; A61F 2310/00383; A61B 5/103; A61F 2310/00592; A61F 2310/00395

(57) ABSTRACT

A method of preparing an interpositional implant suitable for a knee. The method includes determining a three-dimensional shape of a tibial surface of the knee. An implant is produced having a superior surface and an inferior surface, with the superior surface adapted to be positioned against a femoral condyle of the knee, and the inferior surface adapted to be positioned upon the tibial surface of the knee. The inferior surface conforms to the three-dimensional shape of the tibial surface. The implant may be inserted into the knee without making surgical cuts on the tibial surface. The tibial surface may include cartilage, or cartilage and bone.

### CROSS-REFERENCE TO RELATED APPLICATIONS

This application is a continuation of U.S. patent application Ser. No. 11/688,340, entitled “Interpositional Joint Implant,” filed Mar. 20, 2007, which in turn claims the benefit of U.S. provisional application 60/784,255, entitled “Interpositional Joint Implant,” filed Mar. 21, 2006.

U.S. patent application Ser. No. 11/688,340 is also a continuation-in-part of U.S. patent application Ser. No. 10/997,407, entitled “Patient Selectable Knee Joint Arthroplasty Devices,” filed Nov. 24, 2004, which in turn is a continuation-in-part of U.S. patent application Ser. No. 10/752,438, entitled “Patient Selectable Knee Arthroplasty Devices,” filed Jan. 5, 2004, which in turn is a continuation-in-part of U.S. patent application Ser. No. 10/724,010, entitled “Patient Selectable Joint Arthroplasty Devices and Surgical Tools Facilitating Increased Accuracy, Speed and Simplicity in Performing Total and Partial Joint Arthroplasty,” filed Nov. 25, 2003, which in turn is a continuation-in-part of U.S. patent application Ser. No. 10/305,652 entitled “Methods and Compositions for Articular Repair,” filed Nov. 27, 2002, which in turn is a continuation-in-part of U.S. patent application Ser. No. 10/160,667, entitled “Methods and Compositions for Articular Resurfacing,” filed May 28, 2002, which in turn claims the benefit of U.S. provisional patent application 60/293,488 entitled “Methods To Improve Cartilage Repair Systems,” filed May 25, 2001, U.S. provisional patent application 60/363,527, entitled “Novel Devices For Cartilage Repair,” filed Mar. 12, 2002, U.S. patent application 60/380,695, entitled “Methods And Compositions for Cartilage Repair,” filed May 14, 2002 and U.S. patent application 60/380,692, entitled “Methods for Joint Repair,” filed May 14, 2002.

U.S. patent application Ser. No. 11/688,340 is also a continuation-in-part of U.S. application Ser. No. 10/681,750, filed Oct. 7, 2003, entitled “Minimally Invasive Joint Implant with 3-Dimensional Geometry Matching the Articular Surfaces,” which in turn claims the benefit of U.S. provisional patent application 60/467,686 filed May 2, 2003 entitled “Joint Implants” and U.S. provisional patent application 60/416,601, entitled “Minimally Invasive Joint Implant with 3-Dimensional Geometry Matching the Articular Surfaces,” filed on Oct. 7, 2002.

U.S. patent application Ser. No. 11/688,340 is also a continuation-in-part of U.S. application Ser. No. 10/681,749, filed Oct. 7, 2003, entitled “Minimally Invasive Joint Implant with 3-Dimensional Geometry Matching the Articular Surfaces.”

U.S. patent application Ser. No. 11/688,340 is also a continuation-in-part of U.S. application Ser. No. 11/671,745, filed Feb. 6, 2007, entitled “Patient Selectable Joint Arthroplasty Devices and Surgical Tools,” which in turn claims the benefit of: U.S. Ser. No. 60/765,592 entitled “Surgical Tools for Performing Joint Arthroplasty,” filed Feb. 6, 2006; U.S. Ser. No. 60/785,168, entitled “Surgical Tools for Performing Joint Arthroplasty,” filed Mar. 23, 2006; and U.S. Ser. No. 60/788,339, entitled “Surgical Tools for Performing Joint Arthroplasty,” filed Mar. 31, 2006

Each of the above-described applications is incorporated herein, in their entireties, by reference.

### TECHNICAL FIELD

The present invention relates to orthopedic methods, systems and devices and more particularly relates to methods, systems and devices for an interpositional joint implant.

### BACKGROUND ART

There are various types of cartilage, e.g., hyaline cartilage and fibrocartilage. Hyaline cartilage is found at the articular surfaces of bones, e.g., in the joints, and is responsible for providing the smooth gliding motion characteristic of moveable joints. Articular cartilage is firmly attached to the underlying bones and measures typically less than 5 mm in thickness in human joints, with considerable variation depending on the joint and the site within the joint.

Adult cartilage has a limited ability of repair; thus, damage to cartilage produced by disease, such as rheumatoid and/or osteoarthritis, or trauma can lead to serious physical deformity and debilitation. Furthermore, as human articular cartilage ages, its tensile properties change. The superficial zone of the knee articular cartilage exhibits an increase in tensile strength up to the third decade of life, after which it decreases markedly with age as detectable damage to type II collagen occurs at the articular surface. The deep zone cartilage also exhibits a progressive decrease in tensile strength with increasing age, although collagen content does not appear to decrease. These observations indicate that there are changes in mechanical and, hence, structural organization of cartilage with aging that, if sufficiently developed, can predispose cartilage to traumatic damage.

Once damage occurs, joint repair can be addressed through a number of approaches. One approach includes the use of matrices, tissue scaffolds or other carriers implanted with cells (e.g., chondrocytes, chondrocyte progenitors, stromal cells, mesenchymal stem cells, etc.). These solutions have been described as a potential treatment for cartilage and meniscal repair or replacement. See, also, International Publications WO 99/51719 to Fofonoff, published Oct. 14, 1999; WO01/91672 to Simon et al., published Dec. 6, 2001; and WO01/17463 to Mannsmann, published Mar. 15, 2001; U.S. Pat. No. 6,283,980 B1 to Vibe-Hansen et al., issued Sep. 4, 2001, U.S. Pat. No. 5,842,477 to Naughton issued Dec. 1, 1998, U.S. Pat. No. 5,769,899 to Schwartz et al. issued Jun. 23, 1998, U.S. Pat. No. 4,609,551 to Caplan et al. issued Sep. 2, 1986, U.S. Pat. No. 5,041,138 to Vacanti et al. issued Aug. 29, 1991, U.S. Pat. No. 5,197,985 to Caplan et al. issued Mar. 30, 1993, U.S. Pat. No. 5,226,914 to Caplan et al. issued Jul. 13, 1993, U.S. Pat. No. 6,328,765 to Hardwick et al. issued Dec. 11, 2001, U.S. Pat. No. 6,281,195 to Rueger et al. issued Aug. 28, 2001, and U.S. Pat. No. 4,846,835 to Grande issued Jul. 11, 1989. However, clinical outcomes with biologic replacement materials such as allograft and autograft systems and tissue scaffolds have been uncertain since most of these materials do not achieve a morphologic arrangement or structure similar to or identical to that of normal, disease-free human tissue it is intended to replace. Moreover, the mechanical durability of these biologic replacement materials remains uncertain.

Usually, severe damage or loss of cartilage is treated by replacement of the joint with a prosthetic material, for example, silicone, e.g. for cosmetic repairs, or metal alloys. See, e.g., U.S. Pat. No. 6,383,228 to Schmotzer, issued May 7, 2002; U.S. Pat. No. 6,203,576 to Afriat et al., issued Mar. 20, 2001; U.S. Pat. No. 6,126,690 to Ateshian, et al., issued Oct. 3, 2000. Implantation of these prosthetic devices is usually associated with loss of underlying tissue and bone without recovery of the full function allowed by the original cartilage and, with some devices, serious long-term complications associated with the loss of significant amount of tissue and bone can include infection, osteolysis and also loosening of the implant.

Further, joint arthroplasties are highly invasive and require surgical resection of the entire articular surface of one or more bones, or a majority thereof. With these procedures, the marrow space is often reamed to fit the stem of the prosthesis. The reaming results in a loss of the patient's bone stock. U.S. Pat. No. 5,593,450 to Scott et al. issued Jan. 14, 1997 discloses an oval domed shaped patella prosthesis. The prosthesis has a femoral component that includes two condyles as articulating surfaces. The two condyles meet to form a second trochlear groove and ride on a tibial component that articulates with respect to the femoral component. A patella component is provided to engage the trochlear groove. U.S. Pat. No. 6,090,144 to Letot et al. issued Jul. 18, 2000 discloses a knee prosthesis that includes a tibial component and a meniscal component that is adapted to be engaged with the tibial component through an asymmetrical engagement.

A variety of materials can be used in replacing a joint with a prosthetic, for example, silicone, e.g. for cosmetic repairs, or suitable metal alloys are appropriate. See, e.g., U.S. Pat. No. 6,443,991 B1 to Running issued Sep. 3, 2002, U.S. Pat. No. 6,387,131 B1 to Miehlke et al. issued May 14, 2002; U.S. Pat. No. 6,383,228 to Schmotzer issued May 7, 2002; U.S. Pat. No. 6,344,059 B1 to Krakovits et al. issued Feb. 5, 2002; U.S. Pat. No. 6,203,576 to Afriat et al. issued Mar. 20, 2001; U.S. Pat. No. 6,126,690 to Ateshian et al. issued Oct. 3, 2000; U.S. Pat. No. 6,013,103 to Kaufman et al. issued Jan. 11, 2000. Implantation of these prosthetic devices is usually associated with loss of underlying tissue and bone without recovery of the full function allowed by the original cartilage and, with some devices, serious long-term complications associated with the loss of significant amounts of tissue and bone can cause loosening of the implant. One such complication is osteolysis. Once the prosthesis becomes loosened from the joint, regardless of the cause, the prosthesis will then need to be replaced. Since the patient's bone stock is limited, the number of possible replacement surgeries is also limited for joint arthroplasty.

As can be appreciated, joint arthroplasties are highly invasive and require surgical resection of the entire, or a majority of the, articular surface of one or more bones involved in the repair. Typically with these procedures, the marrow space is fairly extensively reamed in order to fit the stem of the prosthesis within the bone. Reaming results in a loss of the patient's bone stock and over time subsequent osteolysis will frequently lead to loosening of the prosthesis. Further, the area where the implant and the bone mate degrades over time requiring the prosthesis to eventually be replaced. Since the patient's bone stock is limited, the number of possible replacement surgeries is also limited for joint arthroplasty. In short, over the course of 15 to 20 years, and in some cases even shorter time periods, the patient can run out of therapeutic options ultimately resulting in a painful, non-functional joint.

U.S. Pat. No. 6,206,927 to Fell, et al., issued Mar. 27, 2001, and U.S. Pat. No. 6,558,421 to Fell, et al., issued May 6, 2003, disclose a surgically implantable knee prosthesis that does not require bone resection. This prosthesis is described as substantially elliptical in shape with one or more straight edges. Accordingly, these devices are not designed to substantially conform to the actual shape (contour) of the remaining cartilage in vivo and/or the underlying bone. Thus, integration of the implant can be extremely difficult due to differences in thickness and curvature between the patient's surrounding cartilage and/or the underlying subchondral bone and the prosthesis. U.S. Pat. No. 6,554,866 to Aicher, et al. issued Apr. 29, 2003 describes a mono-condylar knee joint prosthesis.

Interpositional knee devices that are not attached to both the tibia and femur have been described. For example, Platt et al. (1969) “Mould Arthroplasty of the Knee,” Journal of Bone and Joint Surgery 51B(1):76-87, describes a hemi-arthroplasty with a convex undersurface that was not rigidly attached to the tibia. Devices that are attached to the bone have also been described. Two attachment designs are commonly used. The McKeever design is a cross-bar member, shaped like a “t” from a top perspective view, that extends from the bone mating surface of the device such that the “t” portion penetrates the bone surface while the surrounding surface from which the “t” extends abuts the bone surface. See McKeever, “Tibial Plateau Prosthesis,” Chapter 7, p. 86. An alternative attachment design is the MacIntosh design, which replaces the “t” shaped fin for a series of multiple flat serrations or teeth. See Potter, “Arthroplasty of the Knee with Tibial Metallic Implants of the McKeever and MacIntosh Design,” Surg. Clins. Of North Am. 49(4): 903-915 (1969).

U.S. Pat. No. 4,502,161 to Wall issued Mar. 5, 1985, describes a prosthetic meniscus constructed from materials such as silicone rubber or Teflon with reinforcing materials of stainless steel or nylon strands. U.S. Pat. No. 4,085,466 to Goodfellow et al. issued Mar. 25, 1978, describes a meniscal component made from plastic materials. Reconstruction of meniscal lesions has also been attempted with carbon-fiber-polyurethane-poly (L-lactide). Leeslag, et al., Biological and Biomechanical Performance of Biomaterials (Christel et al., eds.) Elsevier Science Publishers B.V., Amsterdam. 1986. pp. 347-352. Reconstruction of meniscal lesions is also possible with bioresorbable materials and tissue scaffolds.

However, currently available interpositional joint devices do not always provide ideal alignment with the articular surfaces and the resultant joint congruity. Poor alignment and poor joint congruity can, for example, lead to instability of the joint.

Thus, there is a need for an interpositional joint implant or implant system that improves the anatomic result of the joint correction procedure by providing surfaces that more closely resemble the natural knee joint anatomy of a patient. Additionally, what is needed is an implant or implant system that provides for an improved functional joint.

### SUMMARY OF THE INVENTION

The present invention provides novel devices and methods for an interpositional implant that replaces a portion of a joint (e.g., such as the meniscus in a knee joint), where the implant(s) achieves an anatomic or near anatomic fit with the surrounding structures and tissues (e.g., subchondral bone and/or cartilage). The to invention also provides for the preparation of an implantation site with a single cut, or a few relatively small cuts. Asymmetrical components can also be provided to improve the anatomic functionality of the repaired joint by providing a solution that closely resembles the natural knee joint anatomy. The improved anatomic results, in turn, leads to an improved functional result for the repaired joint.

In accordance with a first embodiment of the invention, an interpositional implant suitable for a knee joint is presented. The implant includes a superior surface arranged to oppose at least a portion of a femur, and an inferior surface arranged to oppose at least a portion of a tibial surface. One or more protrusions extend outwardly from the inferior surface. The protrusion has, at its lowest surface, a taper in an anterior to posterior direction.

In accordance with related embodiments of the invention, the taper may extend outwardly a distance from the inferior surface, the distance decreasing moving in the anterior to posterior direction. The protrusion may extend a maximum distance outwardly from the inferior surface at a position anterior to the coordinate system origin. The protrusion may be a keel or a cross-member. The one or more protrusions may include a plurality of protrusions which may be positioned on the inferior surface to be symmetrical, asymmetrical, rows, or random. The one or more protrusions may be adapted to be inserted into one or more cuts made in the tibial surface, such that motion of the implant is limited. The implant may have a substantially U-shaped cross-section in at least one of a medial-lateral direction and an anterior-posterior direction. The superior surface may have a may have a three-dimensional shape that substantially conforms to the tibial surface.

In accordance with further related embodiments of the invention, the superior surface and the inferior surface face opposing directions and define a thickness, The implant includes a peripheral edge extending between the superior and inferior surfaces, with the greatest thickness at the peripheral edge at least 2 mm more than the smallest thickness within the implant. In other embodiments, the thickness of the peripheral edge may be at least 3 mm more than the smallest thickness within the implant.

In accordance with another embodiment of the invention, an implant for insertion in a joint between a first articular surface and a second articular surface is presented. The implant includes a first implant surface that engages with, and substantially conforms to, the first articular surface. The implant further includes a second implant surface for engaging the second articular surface. The second surface is substantially smooth in areas adapted to engage the second articular surface, permitting movement of the second articular surface. The first articular surface includes cartilage.

In accordance with related embodiments of the invention, the first articular surface may include both cartilage and bone. The first implant surface may substantially conforms to the first articular surface such that movement of the implant in the joint is limited. The first implant surface may be adapted to substantially remain fixed to the first articular surface upon a load being placed on the second implant surface. Movement of the implant in the joint may be limited without the use of pins, anchors and adhesives.

In accordance with further related embodiments of the invention, the first articular surface may be a tibial surface and the second articular surface may be a femoral surface. The first implant surface may be substantially concave or substantially convex. The second implant surface may be substantially concave, substantially convex or substantially flat. The second implant surface may be substantially free of irregularities, roughness, and projections in areas which are adapted to contact the second articular surface. The implant may have a substantially U-shaped cross-section in at least one of a medial-lateral direction and an anterior-posterior direction.

In accordance with another embodiment of the invention, an implant for insertion in a joint between a first articular surface and a second articular surface is presented. The implant includes a first implant surface for engaging the first articular surface. The first implant surface has one or more convexities and one or more concavities. A second implant surface engages the second articular surface, the second implant surface having at least one of a plurality of concavities and a plurality of convexities.

In accordance with related embodiments of the invention, the first articular surface may be a tibial surface, and the second articular surface may be a femoral surface. The first articular surface may include cartilage, or both cartilage and bone. The first implant surface may substantially conform to the first articular surface such that movement of the implant in the joint is limited. The first implant surface may be adapted to substantially remain fixed to the first articular surface upon a load being placed on the second implant surface. Movement of the implant in the joint may be limited without the use of pins, anchors and adhesives. The second surface may be substantially smooth in areas adapted to engage the second articular surface, permitting movement of the second articular surface. The second implant surface may be substantially free of irregularities, roughness, and projections in areas which are adapted to contact the second articular surface. The joint may be a hip joint, ankle joint, toe joint, shoulder joint, elbow joint, wrist joint, or finger joint.

In accordance with another embodiment of the invention, an implant for insertion in a knee joint between a tibial articular surface and a femoral articular surface is presented. The implant includes a first implant surface for engaging the tibial articular surface, and a second implant surface for engaging the femoral articular surface. The second implant surface has a plurality of concavities.

In accordance with related embodiments of the invention, the second implant surface may also has a plurality of convexities. The first implant surface may have one or more convexities and one or more concavities. The first implant surface may substantially conform to the tibial articular surface, such that, for example, movement of the implant in the joint is limited. The first implant surface may be adapted to substantially remain fixed to the first articular surface upon a load being placed on the second implant surface. Movement of the implant in the joint may be limited without the use of pins, anchors and adhesives.

In accordance with another embodiment of the invention, an implant for insertion in a knee joint between a tibial articular surface and a femoral articular surface is presented. The implant includes a first implant surface for engaging the femoral articular surface, and a second implant surface for engaging the tibial articular surface. The second implant surface has a plurality of convexities.

In accordance with related embodiments of the invention, the second implant surface may also has a plurality of concavities. The first implant surface may have one or more convexities and one or more concavities. The second implant surface may substantially conform to the tibial articular surface, such that, for example, movement of the implant in the joint is limited. The second implant surface may be adapted to substantially remain fixed to the tibial articular surface upon a load being placed on the second implant surface. Movement of the implant in the joint may be limited without the use of pins, anchors and adhesives.

In accordance with further related embodiments of the invention, the tibial articular surface may include cartilage, or cartilage and bone. The second implant surface may be substantially smooth in areas adapted to engage the femoral articular surface, permitting movement of the femoral articular surface. The second implant surface may be substantially free of irregularities, roughness, and projections in areas which are adapted to contact the femoral articular surface.

In accordance with another embodiment of the invention, an implant is presented for insertion in a joint having a first articular surface. The first articular surface includes cartilage. The implant includes a first implant surface conforming to the first articular surface.

In accordance with related embodiments of the invention, the first articular surface may further include bone. The joint may have a second articular surface, with the implant for insertion between the first articular surface and the second articular surface. The implant may further include a second implant surface for engaging the second articular surface.

In accordance with further related embodiments of the invention, the second surface may be substantially smooth in areas adapted to engage the second articular surface, permitting movement of the second articular surface. The second implant surface may be substantially free of irregularities, roughness, and projections in areas which are adapted to contact the second articular surface.

In accordance with still further related embodiments of the invention, the first articular surface may be a tibial surface, and the second articular surface may be a femoral surface. The first implant surface may substantially conform to the first articular surface such that movement of the implant in the joint is limited. The first implant surface may be adapted to substantially remain fixed to the first articular surface upon a load being placed on the second implant surface. Movement of the implant in the joint may be limited without the use of pins, anchors and adhesives. The joint is one of a hip joint, ankle joint, toe joint, shoulder joint, elbow joint, wrist joint, or a finger joint.

In accordance with another embodiment of the invention, an interpositional implant suitable for a knee joint is presented. The implant includes a superior surface arranged to oppose at least a portion of a femur, and an inferior surface arranged to oppose at least a portion of a tibial surface. The implant has a substantially U-shaped cross-section in at least one of a medial-lateral direction and an anterior-posterior direction. In related embodiments, the superior surface has a substantially U-shaped cross-section in the medial-lateral direction.

In accordance with another embodiment of the invention, an interpositional implant suitable for a knee joint is presented. The implant includes a superior surface arranged to oppose at least a portion of a femur, and an inferior surface arranged to oppose at least a portion of a tibial surface. The implant has a substantially inverted U-shaped cross-section in at least one of a medial-lateral direction and an anterior-posterior direction.

In accordance with another embodiment of the invention, an interpositional implant suitable for a knee joint is presented. The implant includes a superior surface arranged to oppose at least a portion of a femur, and an inferior surface arranged to oppose at least a portion of a tibial surface. The implant has a substantially inverted U-shaped cross-section in a medial-lateral direction.

In accordance with another embodiment of the invention, an interpositional implant suitable for a knee joint is presented. The implant includes a superior surface arranged to contact at least a portion of a femur, and an inferior surface arranged to contact at least a portion of a tibial surface. The superior surface and the inferior surface face opposing directions and defining a thickness. A peripheral edge extends between the superior and inferior surfaces, the greatest thickness at the peripheral edge at least 2 mm more than the smallest thickness of the implant. In related embodiments of the invention, the greatest thickness at the peripheral edge is at least one of 3 mm, 4 mm, 5 mm, 6 mm and 7 mm more than the smallest thickness within the implant.

In accordance with another embodiment of the invention, an interpositional implant suitable for a knee joint is presented. The implant includes a superior surface arranged to contact at least a portion of a femur, and an inferior surface arranged to contact at least a portion of a tibial surface. The superior surface and the inferior surface face opposing directions, with the superior surface having a height relative to the inferior surface at its lowest point. A peripheral edge extends between the superior and inferior surfaces. The greatest height at the peripheral edge is greater than the smallest height within the implant by a ratio of 2:1. In related embodiment of the invention, the peripheral edge may have a height that is greater than the smallest height within the implant by a ratio of one of 3:1, 4:1 and 5:1.

In accordance with another embodiment of the invention, an interpositional implant suitable for a knee joint is presented. The implant includes a superior surface arranged to contact at least a portion of a femur, and an inferior surface arranged to contact at least a portion of a tibial surface. A peripheral edge extends between the superior and inferior surfaces, the peripheral edge having a varying center that defines a perimeter around the implant, wherein a lowest point of a central portion of the superior surface is lower than 30% of the perimeter. In accordance with related embodiments of the invention, the lowest point of the central portion of the superior surface is lower than 40% or 50% of the perimeter.

In accordance with another embodiment of the invention, an implant is presented for insertion in a joint between a first articular surface and a second articular surface. A first implant surface conforms to the first articular surface, the first articular surface including cartilage. The first implant surface has a periphery, the periphery including a stabilization mechanism for limiting motion of the implant in the joint. The implant further includes a second implant surface for contacting the second articular surface.

In accordance with related embodiments of the invention, the first articular surface may further include bone. The stabilization mechanism may be a ridge, a lip or a thickening. The stabilization mechanism may be located along a portion of the periphery. For example, the stabilization mechanism may engage the tibial spine. The stabilization mechanism may engage a peripheral edge of the first articular surface. The stabilization mechanism may include at least one of a concavity and a convexity.

In accordance with further related embodiments of the invention, the first articular surface may be a tibial surface, and the second articular surface may be a femoral surface. The first implant surface may substantially conform to the shape of tibial surface. The second implant surface may be substantially smooth in areas adapted to engage the second articular surface. The second implant surface may allow movement of the second articular surface.

In accordance with another embodiment of the invention, a method of making an interpositional implant suitable for a knee is presented. The method includes producing an implant having a superior surface and an inferior surface. The superior surface is adapted to be positioned against a femoral condyle of the knee, and the inferior surface is adapted to be positioned upon and conform to the tibial surface of the knee. The tibial surface includes cartilage. The inferior surface has a periphery, the periphery including a stabilization mechanism for restricting motion of the implant in the joint.

In accordance with related embodiments of the invention, the tibial surface may further include bone. The stabilization mechanism may be a ridge, a lip or a thickening. The stabilization mechanism may be located along a portion of the periphery. The stabilization mechanism may engage the tibial spine. The stabilization mechanism may engage a peripheral edge of the tibial surface.

In accordance with another embodiment of the invention, an implant is interposed in a joint between a first articular surface and a second articular surface. The implant includes a first surface for contacting the first articular surface such that motion of the implant is constrained. The implant further includes a second surface for contacting the second articular surface, the second surface allowing movement of the second articular surface.

In accordance with related embodiments of the invention, the first surface may substantially conform to the first articular surface such that movement of the implant in the joint is limited. The first articular surface may include cartilage, or cartilage and bone. The first surface may be adapted to substantially remain fixed to the first articular surface upon a load being placed on the second surface. Movement of the implant in the joint may be constrained without the use of pins, anchors and adhesives.

In accordance with further related embodiments of the invention, the first articular surface may be a tibial surface and the second articular surface may be a femoral surface. The first surface may include one or more shapes selected from the group consisting of substantially concave and substantially convex. The second surface may be one of substantially concave, substantially convex, and substantially flat. The second surface may be substantially free of irregularities, roughness, and projections in areas that are adapted to contact the second articular surface. The implant may have a substantially U-shaped cross-section in at least one of a medial-lateral direction and an anterior-posterior direction.

In accordance with another embodiment of the invention, a method of preparing an interpositional implant suitable for a knee is presented. The method includes determining a three-dimensional shape of a tibial surface of the knee. An implant is produced having a superior surface and an inferior surface, with the superior surface adapted to be positioned against a femoral condyle of the knee, and the inferior surface adapted to be positioned upon the tibial surface of the knee. The inferior surface conforms to the three-dimensional shape of the tibial surface.

In accordance with another embodiment of the invention, the implant is inserted into the knee without making surgical cuts on the tibial surface. The tibial surface may include cartilage. The tibial surface may further include bone.

### BRIEF DESCRIPTION OF THE DRAWINGS

The foregoing features of the invention will be more readily understood by reference to the following detailed description, taken with reference to the accompanying drawings, in which:

FIG. 1A is a block diagram of a method for assessing a joint in need of repair according to the invention wherein the existing joint surface is unaltered, or substantially unaltered, prior to receiving the selected implant. FIG. 1B is a block diagram of a method for assessing a joint in need of repair according to the invention wherein the existing joint surface is unaltered, or substantially unaltered, prior to designing an implant suitable to achieve the repair. FIG. 1C is a block diagram of a method for developing an implant and using the implant in a patient.

FIG. 2A is a perspective view of a joint implant of the invention suitable for implantation at the tibial plateau of the knee joint. FIG. 2B is a top view of the implant of FIG. 2A. FIG. 2C is a cross-sectional view of the implant of FIG. 2B along the lines C-C shown in FIG. 2B. FIG. 2D is a cross-sectional view along the lines D-D shown in FIG. 2B. FIG. 2E is a cross-sectional view along the lines E-E shown in FIG. 2B. FIG. 2F is a side view of the implant of FIG. 2A. FIG. 2G is a cross-sectional view of the implant of FIG. 2A shown implanted taken along a plane parallel to the sagittal plane. FIG. 2H is a cross-sectional view of the implant of FIG. 2A shown implanted taken along a plane parallel to the coronal plane. FIG. 2I is a cross-sectional view of the implant of FIG. 2A shown implanted taken along a plane parallel to the axial plane. FIG. 2J shows a slightly larger implant that extends closer to the bone medially (towards the edge of the tibial plateau) and anteriorly and posteriorly. FIG. 2K is a side view of an alternate embodiment of the joint implant of FIG. 2A showing an anchor in the form of a keel. FIG. 2L is a bottom view of an alternate embodiment of the joint implant of FIG. 2A showing an anchor. FIG. 2M shows an anchor in the form of a cross-member. FIGS. 2N-1, 2N-2, 2O-1 and 2O-2 are alternative embodiments of the implant showing the lower surface have a trough for receiving a cross-bar. FIG. 2P illustrates a variety of cross-bars. FIGS. 2Q-R illustrate the device implanted within a knee joint. FIGS. 2S(1-9) illustrate another implant suitable for the tibial plateau further having a chamfer cut along one edge. FIG. 2T(1-8) illustrate an alternate embodiment of the tibial implant wherein the surface of the joint is altered to create a flat or angled surface for the implant to mate with.

### DETAILED DESCRIPTION OF THE INVENTION

The following description is presented to enable any person skilled in the art to make and use the invention. Various modifications to the embodiments described will be readily apparent to those skilled in the art, and the generic principles defined herein can be applied to other embodiments and applications without departing from the spirit and scope of the present invention as defined by the appended claims. Thus, the present invention is not intended to be limited to the embodiments shown, but is to be accorded the widest scope consistent with the principles and features disclosed herein. To the extent necessary to achieve a complete understanding of the invention disclosed, the specification and drawings of all issued patents, patent publications, and patent applications cited in this application are incorporated herein by reference.

As will be appreciated by those of skill in the art, methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The practice of the present invention can employ, unless otherwise indicated, conventional and digital methods of x-ray imaging and processing, x-ray tomosynthesis, ultrasound including A-scan, B-scan and C-scan, computed tomography (CT scan), magnetic resonance imaging (MRI), optical coherence tomography, single photon emission tomography (SPECT) and positron emission tomography (PET) within the skill of the art. Such techniques are explained fully in the literature and need not be described herein. See, e.g., X-Ray Structure Determination: A Practical Guide, 2nd Edition, editors Stout and Jensen, 1989, John Wiley & Sons, publisher; Body CT: A Practical Approach, editor Slone, 1999, McGraw-Hill publisher; X-ray Diagnosis: A Physician's Approach, editor Lam, 1998 Springer-Verlag, publisher; and Dental Radiology: Understanding the X-Ray Image, editor Laetitia Brocklebank 1997, Oxford University Press publisher. See also, The Essential Physics of Medical Imaging (2^{nd} Ed.), Jerrold T. Bushberg, et al.

The present invention provides methods and compositions for repairing a joint, and more particularly for an interpositional knee implant for implantation at the tibial plateau. Among other things, the techniques described herein allow for the customization of the interpositional joint implant to a joint of a particular subject, for example in terms of size, thickness and/or curvature. By forming the shape (e.g., size, thickness and/or curvature) of the interpositional joint implant to be an exact or near anatomic fit with the underlying joint surface minimizes the need for bone removal, and the success of repair is enhanced. The repair material can be shaped prior to implantation and such shaping can be based, for example, on electronic images that provide information regarding curvature or thickness of underlying subchondral bone and/or cartilage. Thus, the current invention provides, among other things, for minimally invasive methods for partial joint replacement. The methods will require only minimal or, in some instances, no loss in bone stock.

Advantages of the present invention can include, but are not limited to, (i) customization of joint repair, thereby enhancing the efficacy and comfort level for the patient following the repair procedure; (ii) eliminating, in some embodiments, the need for a surgeon to measure the joint intraoperatively; (iii) eliminating the need for a surgeon to shape the material during the implantation procedure; (iv) providing methods of evaluating curvature of the repair material based on bone or tissue images or based on intraoperative probing techniques; (v) providing methods of repairing joints with only minimal or, in some instances, no loss in bone stock; (vi) improving postoperative joint congruity; (vii) improving the postoperative patient recovery in some embodiments and (viii) improving postoperative function, such as range of motion.

Thus, the methods described herein allow for the design and use of an interpositional joint implant that more precisely fits the articular surface(s) and, accordingly, provides improved repair of the joint.

I. Assessment of Joints and Alignment

The methods and compositions described herein can be used to treat defects resulting from disease of the cartilage (e.g., osteoarthritis), bone damage, cartilage damage, trauma, and/or degeneration due to overuse or age. The invention allows, among other things, a health practitioner to evaluate and treat such defects.

As will be appreciated by those of skill in the art, size, curvature and/or thickness measurements can be obtained using any suitable technique. For example, one-dimensional, two-dimensional, and/or three-dimensional measurements can be obtained using suitable mechanical means, laser devices, electromagnetic or optical tracking systems, molds, materials applied to the articular surface that harden and “memorize the surface contour,” and/or one or more imaging techniques known in the art. Measurements can be obtained non-invasively and/or intraoperatively (e.g., using a probe or other surgical device). As will be appreciated by those of skill in the art, the thickness of the repair device can vary at any given point depending upon patient's anatomy and/or the depth of the damage to the cartilage and/or bone to be corrected at any particular location on an articular surface.

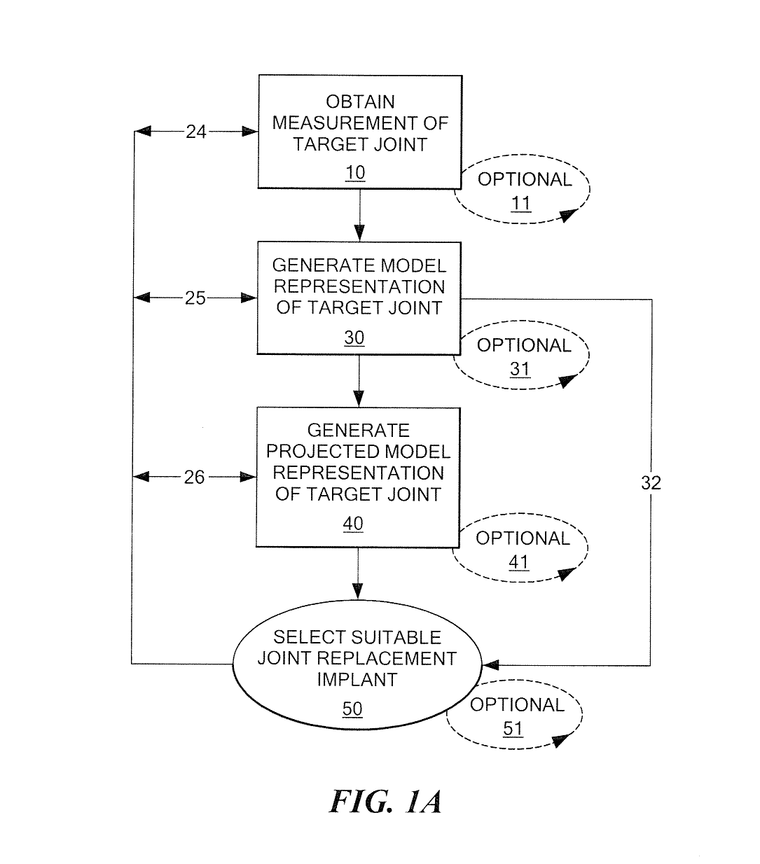

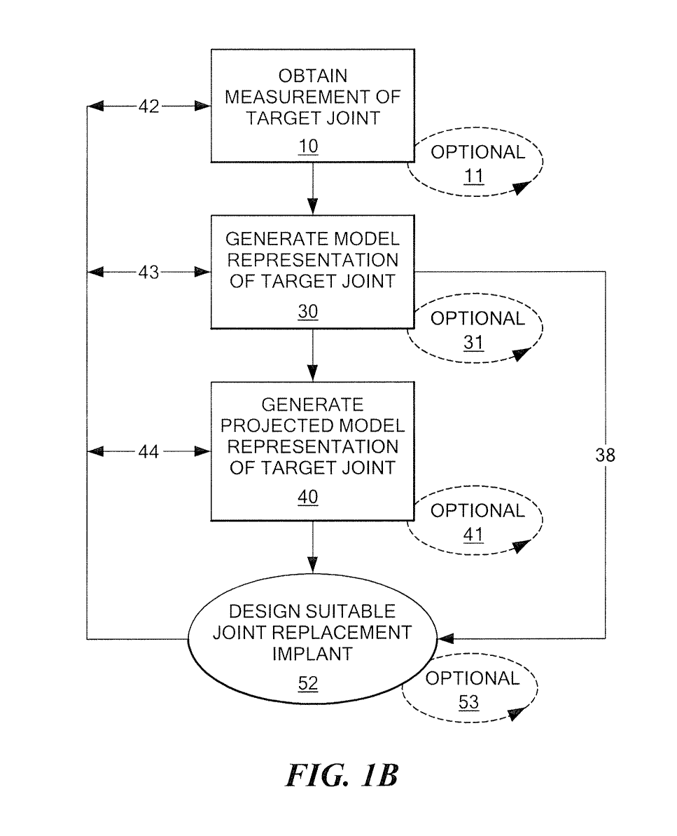

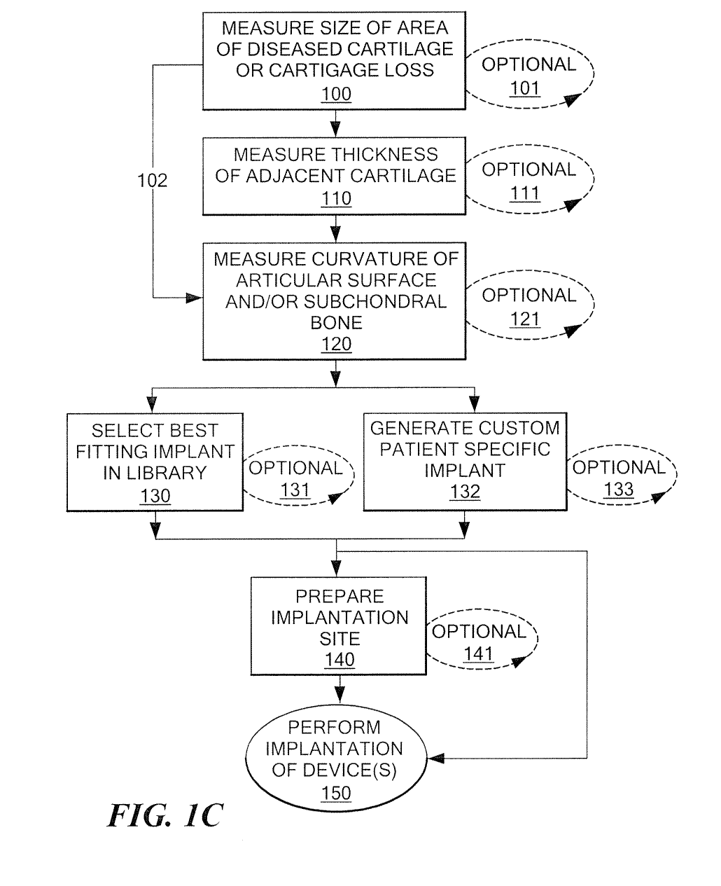

FIG. 1A is a flow chart showing steps taken by a practitioner in assessing a joint. First, a practitioner obtains a measurement of a target joint 10. The step of obtaining a measurement can be accomplished by taking an image of the joint. This step can be repeated, as necessary, 11 to obtain a plurality of images in order to further refine the joint assessment process. Once the practitioner has obtained the necessary measurements, the information is used to generate a model representation of the target joint being assessed 30. This model representation can be in the form of a topographical map or image. The model representation of the joint can be in one, two, or three dimensions. It can include a physical model. More than one model can be created 31, if desired. Either the original model, or a subsequently created model, or both can be used. After the model representation of the joint is generated 30, the practitioner can optionally generate a projected model representation of the target joint in a corrected condition 40, e.g., from the existing cartilage on the joint surface, by providing a mirror of the opposing joint surface, or a combination thereof Again, this step can be repeated 41, as necessary or desired. Using the difference between the topographical condition of the joint and the projected image of the joint, the practitioner can then select a joint implant 50 that is suitable to achieve the corrected joint anatomy. As will be appreciated by those of skill in the art, the selection process 50 can be repeated 51 as often as desired to achieve the desired result. Additionally, it is contemplated that a practitioner can obtain a measurement of a target joint 10 by obtaining, for example, an x-ray, and then select a suitable joint replacement implant 50.

As will be appreciated by those of skill in the art, the practitioner can proceed directly from the step of generating a model representation of the target joint 30 to the step of selecting a suitable joint replacement implant 50 as shown by the arrow 32. Additionally, following selection of suitable joint replacement implant 50, the steps of obtaining measurement of target joint 10, generating model representation of target joint 30 and generating projected model 40, can be repeated in series or parallel as shown by the flow 24, 25, 26.

FIG. 1B is an alternate flow chart showing steps taken by a practitioner in assessing a joint. First, a practitioner obtains a measurement of a target joint 10. The step of obtaining a measurement can be accomplished by taking an image of the joint. This step can be repeated, as necessary, 11 to obtain a plurality of images in order to further refine the joint assessment process. Once the practitioner has obtained the necessary measurements, the information is used to generate a model representation of the target joint being assessed 30. This model representation can be in the form of a topographical map or image. The model representation of the joint can be in one, two, or three dimensions. The process can be repeated 31 as necessary or desired. It can include a physical model. After the model representation of the joint is assessed 30, the practitioner can optionally generate a projected model representation of the target joint in a corrected condition 40. This step can be repeated 41 as necessary or desired. Using the difference between the topographical condition of the joint and the projected image of the joint, the practitioner can then design a joint implant 52 that is suitable to achieve the corrected joint anatomy, repeating the design process 53 as often as necessary to achieve the desired implant design. The practitioner can also assess whether providing additional features, such as rails, keels, lips, pegs, cruciate stems, or anchors, cross-bars, etc. will enhance the implants' performance in the target joint.

As will be appreciated by those of skill in the art, the practitioner can proceed directly from the step of generating a model representation of the target joint 30 to the step of designing a suitable joint replacement implant 52 as shown by the arrow 38. Similar to the flow shown above, following the design of a suitable joint replacement implant 52, the steps of obtaining measurement of target joint 10, generating model representation of target joint 30 and generating projected model 40, can be repeated in series or parallel as shown by the flow 42, 43, 44.

FIG. 1C is a flow chart illustrating the process of selecting an implant for a patient. First, using the techniques described above or those suitable and known in the art at the time the invention is practiced, the size of area of diseased cartilage or cartilage loss may be measured 100. This step can be repeated multiple times 101, as desired. The thickness of adjacent cartilage can optionally be measured 110. This process can also be repeated as desired 111. The curvature of the underlying articular surface and/or subchondral bone is then measured 120. As will be appreciated measurements can be taken of the surface of the joint being repaired, or of the mating surface in order to facilitate development of the best design for the implant surface.

Once the surfaces have been measured, the user either selects the best fitting implant contained in a library of implants 130 or generates a patient-specific implant 132. These steps can be repeated as desired or necessary to achieve the best fitting implant for a patient, 131, 133. As will be appreciated by those of skill in the art, the process of selecting or designing an implant can be tested against the information contained in the MRI or x-ray of the patient to ensure that the surfaces of the device achieves a good fit relative to the patient's joint surface. Testing can be accomplished by, for example, superimposing the implant image over the image for the patient's joint. Once it has been determined that a suitable implant has been selected or designed, the implant site can be prepared 140, for example by removing cartilage or bone from the joint surface, or the implant can be placed into the joint 150.

The joint implant selected or designed achieves anatomic or near anatomic fit with the existing surface of the joint while presenting a mating surface for the opposing joint surface that replicates the natural joint anatomy. In this instance, both the existing surface of the joint can be assessed as well as the desired resulting surface of the joint. This technique is particularly useful for implants that are not anchored into the bone.

As will be appreciated by those of skill in the art, the physician, or other person practicing the invention, can obtain a measurement of a target joint 10 and then either design 52 or select 50 a suitable joint replacement implant.

II. Repair Materials

A wide variety of materials find use in the practice of the present invention, including, but not limited to, plastics, metals, crystal free metals, ceramics, biological materials (e.g., collagen or other extracellular matrix materials), hydroxyapatite, cells (e.g., stem cells, chondrocyte cells or the like), or combinations thereof. Based on the information (e.g., measurements) obtained regarding, for example, the articular surface and/or the subchondral bone, a repair material can be formed or selected. Further, using one or more of these techniques described herein, the interpositional knee implant may be designed or selected that has a curvature that will fit the contour and shape of the articular surface and/or subchondral bone. The repair material can include any combination of materials, and typically includes at least one non-pliable material. For example, the repair material may be inflexible, and/or not easily bent or changed.

A. Metal and Polymeric Repair Materials

Currently, joint repair systems often employ metal and/or polymeric materials including, for example, prostheses which are anchored into the underlying bone (e.g., a tibia in the case of a knee prosthesis). See, e.g., U.S. Pat. No. 6,203,576 to Afriat, et al. issued Mar. 20, 2001 and U.S. Pat. No. 6,322,588 to Ogle, et al. issued Nov. 27, 2001, and references cited therein. A wide-variety of metals are useful in the practice of the present invention, and can be selected based on any criteria. For example, material selection can be based on resiliency to impart a desired degree of rigidity. Non-limiting examples of suitable metals include silver, gold, platinum, palladium, iridium, copper, tin, lead, antimony, bismuth, zinc, titanium, cobalt, stainless steel, nickel, iron alloys, cobalt alloys, such as Elgiloy®, a cobalt-chromium-nickel alloy, and MP35N, a nickel-cobalt-chromium-molybdenum alloy, and Nitinol™, a nickel-titanium alloy, aluminum, manganese, iron, tantalum, crystal free metals, such as Liquidmetal® alloys (available from LiquidMetal Technologies, www.liquidmetal.com), other metals that can slowly form polyvalent metal ions, for example to inhibit calcification of implanted substrates in contact with a patient's bodily fluids or tissues, and combinations thereof.

Suitable synthetic polymers include, without limitation, polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, similar copolymers and mixtures thereof. Bioresorbable synthetic polymers can also be used such as dextran, hydroxyethyl starch, derivatives of gelatin, polyvinylpyrrolidone, polyvinyl alcohol, poly[N-(2-hydroxypropyl) methacrylamide], poly(hydroxy acids), poly(epsilon-caprolactone), polylactic acid, polyglycolic acid, poly(dimethyl glycolic acid), poly(hydroxy butyrate), and similar copolymers can also be used.

Other materials would also be appropriate, for example, the polyketone known as polyetheretherketone (PEEK™). This includes the material PEEK 450G, which is an unfilled PEEK approved for medical implantation available from Victrex of Lancashire, Great Britain. (Victrex is located at www.matweb.com or see Boedeker www.boedeker.com). Other sources of this material include Gharda located in Panoli, India (www.ghardapolymers.com).

It should be noted that the material selected can also be filled. For example, other grades of PEEK are also available and contemplated, such as 30% glass-filled or 30% carbon filled, provided such materials are cleared for use in implantable devices by the FDA, or other regulatory body. Glass filled PEEK reduces the expansion rate and increases the flexural modulus of PEEK relative to that portion which is unfilled. The resulting product is known to be ideal for improved strength, stiffness, or stability. Carbon filled PEEK is known to enhance the compressive strength and stiffness of PEEK and lower its expansion rate. Carbon filled PEEK offers wear resistance and load carrying capability.

As will be appreciated, other suitable similarly biocompatible thermoplastic or thermoplastic polycondensate materials that resist fatigue, have good memory, are inflexible or flexible, have very low moisture absorption, and good wear and/or abrasion resistance, can be used without departing from the scope of the invention. The implant can also be comprised of polyetherketoneketone (PEKK).

Other materials that can be used include polyetherketone (PEK), polyetherketoneetherketoneketone (PEKEKK), and polyetheretherketoneketone (PEEKK), and generally a polyaryletheretherketone. Further other polyketones can be used as well as other thermoplastics.

Reference to appropriate polymers that can be used for the implant can be made to the following documents, all of which are incorporated herein by reference. These documents include: PCT Publication WO 02/02158 A1, dated Jan. 10, 2002 and entitled Bio-Compatible Polymeric Materials; PCT Publication WO 02/00275 A1, dated Jan. 3, 2002 and entitled Bio-Compatible Polymeric Materials; and PCT Publication WO 02/00270 A1, dated Jan. 3, 2002 and entitled Bio-Compatible Polymeric Materials.

The polymers can be prepared by any of a variety of approaches including conventional polymer processing methods. Preferred approaches include, for example, injection molding, which is suitable for the production of polymer components with significant structural features, and rapid prototyping approaches, such as reaction injection molding and stereo-lithography. The substrate can be textured or made porous by either physical abrasion or chemical alteration to facilitate incorporation of the metal coating. Other processes are also appropriate, such as extrusion, injection, compression molding and/or machining techniques. Typically, the polymer is chosen for its physical and mechanical properties and is suitable for carrying and spreading the physical load between the joint surfaces.

More than one metal and/or polymer can be used in combination with each other. For example, one or more metal-containing substrates can be coated with polymers in one or more regions or, alternatively, one or more polymer-containing substrate can be coated in one or more regions with one or more metals.

The system or prosthesis can be porous or porous coated. The porous surface components can be made of various materials including metals, ceramics, and polymers. These surface components can, in turn, be secured by various means to a multitude of structural cores formed of various metals. Suitable porous coatings include, but are not limited to, metal, ceramic, polymeric (e.g., biologically neutral elastomers such as silicone rubber, polyethylene terephthalate and/or combinations thereof) or combinations thereof. See, e.g., U.S. Pat. No. 3,605,123 to Hahn, issued Sep. 20, 1971. U.S. Pat. No. 3,808,606 to Tronzo issued May 7, 1974 and U.S. Pat. No. 3,843,975 to Tronzo issued Oct. 29, 1974; U.S. Pat. No. 3,314,420 to Smith issued Apr. 18, 1967; U.S. Pat. No. 3,987,499 to Scharbach issued Oct. 26, 1976; and German Offenlegungsschrift 2,306,552. There can be more than one coating layer and the layers can have the same or different porosities. See, e.g., U.S. Pat. No. 3,938,198 to Kahn, et al., issued Feb. 17, 1976.

The coating can be applied by surrounding a core with powdered polymer and heating until cured to form a coating with an internal network of interconnected pores. The tortuosity of the pores (e.g., a measure of length to diameter of the paths through the pores) can be important in evaluating the probable success of such a coating in use on a prosthetic device. See, also, U.S. Pat. No. 4,213,816 to Morris issued Jul. 22, 1980. The porous coating can be applied in the form of a powder and the article as a whole subjected to an elevated temperature that bonds the powder to the substrate. Selection of suitable polymers and/or powder coatings can be determined in view of the teachings and references cited herein, for example based on the melt index of each.

B. Biological Repair Material

Repair materials can also include one or more biological material either alone or in combination with non-biological materials. For example, any base material can be designed or shaped and suitable cartilage replacement or regenerating material(s) such as fetal cartilage cells can be applied to be the base. The cells can be then be grown in conjunction with the base until the desired thickness (and/or curvature) is reached. Conditions for growing cells (e.g., chondrocytes) on various substrates in culture, ex vivo and in vivo are described, for example, in U.S. Pat. No. 5,478,739 to Slivka et al. issued Dec. 26, 1995; U.S. Pat. No. 5,842,477 to Naughton et al. issued Dec. 1, 1998; U.S. Pat. No. 6,283,980 to Vibe-Hansen et al., issued Sep. 4, 2001, and U.S. Pat. No. 6,365,405 to Salzmann et al. issued Apr. 2, 2002. Non-limiting examples of suitable substrates include plastic, tissue scaffold, a bone replacement material (e.g., a hydroxyapatite, a bioresorbable material), or any other material suitable for growing a cartilage replacement or regenerating material on it.

Biological polymers can be naturally occurring or produced in vitro by fermentation and the like. Suitable biological polymers include, without limitation, collagen, elastin, silk, keratin, gelatin, polyamino acids, cat gut sutures, polysaccharides (e.g., cellulose and starch) and mixtures thereof. Biological polymers can be bioresorbable.

Biological materials used in the methods described herein can be autografts (from the same subject); allografts (from another individual of the same species) and/or xenografts (from another species). See, also, International Patent Publications WO 02/22014 to Alexander et al. published Mar. 21, 2002 and WO 97/27885 to Lee published Aug. 7, 1997. In certain embodiments autologous materials are preferred, as they can carry a reduced risk of immunological complications to the host, including re-absorption of the materials, inflammation and/or scarring of the tissues surrounding the implant site.

In one embodiment of the invention, a probe is used to harvest tissue from a donor site and to prepare a recipient site. The donor site can be located in a xenograft, an allograft or an autograft. The probe is used to achieve a good anatomic match between the donor tissue sample and the recipient site. The probe is specifically designed to achieve a seamless or near seamless match between the donor tissue sample and the recipient site. The probe can, for example, be cylindrical. The distal end of the probe is typically sharp in order to facilitate tissue penetration. Additionally, the distal end of the probe is typically hollow in order to accept the tissue. The probe can have an edge at a defined distance from its distal end, e.g. at 1 cm distance from the distal end and the edge can be used to achieve a defined depth of tissue penetration for harvesting. The edge can be external or can be inside the hollow portion of the probe. For example, an orthopedic surgeon can take the probe and advance it with physical pressure into the cartilage, the subchondral bone and the underlying marrow in the case of a joint such as a knee joint. The surgeon can advance the probe until the external or internal edge reaches the cartilage surface. At that point, the edge will prevent further tissue penetration thereby achieving a constant and reproducible tissue penetration. The distal end of the probe can include one or more blades, saw-like structures, or tissue cutting mechanism. For example, the distal end of the probe can include an iris-like mechanism consisting of several small blades. The blade or blades can be moved using a manual, motorized or electrical mechanism thereby cutting through the tissue and separating the tissue sample from the underlying tissue. Typically, this will be repeated in the donor and the recipient. In the case of an iris-shaped blade mechanism, the individual blades can be moved so as to close the iris thereby separating the tissue sample from the donor site.

In another embodiment of the invention, a laser device or a radiofrequency device can be integrated inside the distal end of the probe. The laser device or the radiofrequency device can be used to cut through the tissue and to separate the tissue sample from the underlying tissue.

In one embodiment of the invention, the same probe can be used in the donor and in the recipient. In another embodiment, similarly shaped probes of slightly different physical dimensions can be used. For example, the probe used in the recipient can be slightly smaller than that used in the donor thereby achieving a tight fit between the tissue sample or tissue transplant and the recipient site. The probe used in the recipient can also be slightly shorter than that used in the donor thereby correcting for any tissue lost during the separation or cutting of the tissue sample from the underlying tissue in the donor material.

Any biological repair material can be sterilized to inactivate biological contaminants such as bacteria, viruses, yeasts, molds, mycoplasmas and parasites. Sterilization can be performed using any suitable technique, for example radiation, such as gamma radiation.

Any of the biological materials described herein can be harvested with use of a robotic device. The robotic device can use information from an electronic image for tissue harvesting.

In certain embodiments, the cartilage replacement material has a particular biochemical composition. For instance, the biochemical composition of the cartilage surrounding a defect can be assessed by taking tissue samples and chemical analysis or by imaging techniques. For example, WO 02/22014 to Alexander describes the use of gadolinium for imaging of articular cartilage to monitor glycosaminoglycan content within the cartilage. The cartilage replacement or regenerating material can then be made or cultured in a manner, to achieve a biochemical composition similar to that of the cartilage associated with the implantation site. The culture conditions used to achieve the desired biochemical compositions can include, for example, varying concentrations. Biochemical composition of the cartilage replacement or regenerating material can, for example, be influenced by controlling concentrations and exposure times of certain nutrients and growth factors.

III. Device Design

In illustrative embodiments of the invention, an interpositional joint implant is presented. The form of the implant or device is determined by projecting the contour of the existing cartilage and/or bone to effectively mimic aspects of the natural articular structure. The device substantially restores the normal joint alignment and/or provides a congruent or substantially congruent surface to the original or natural articular surface of an opposing joint surface that it mates with. Further, it can essentially eliminate further degeneration because the conforming surfaces of the device provide an anatomic or near anatomic fit with the existing articular surfaces of the joint. Insertion of the device is done via a small (e. g., 3 cm to 5 cm) incision and no bone resection or mechanical fixation of the device is required. However, as will be appreciated by those of skill in the art, additional structures can be provided, such as a cross-bar, fins, pegs, teeth (e.g., pyramidal, triangular, spheroid, or conical protrusions), or pins, that enhance the devices' ability to seat more effectively on the joint surface. Osteophytes or other structures that interfere with the device placement are easily removed. By occupying the joint space in an anatomic or near anatomic fit, the device improves joint stability and restores normal or near normal mechanical alignment of the joint.

The precise dimensions of the devices described herein can be determined by obtaining and analyzing images of a particular subject and designing a device that substantially conforms to the subject's joint anatomy (e.g., cartilage, bone, or cartilage and bone) while taking into account the existing articular surface anatomy as described above. Thus, the actual shape of the present device can be tailored to the individual.

A prosthetic device of the subject invention can be a device suitable for minimally invasive, surgical implantation without requiring bone resection. The device may be generally self-centering, and/or use various anchoring/stabilization mechanisms. In various embodiments, the device may include a surface that conforms and mates with the opposing joint surface (e.g., cartilage and/or subchondral bone), such that movement of the device is limited without the use pins, anchors and/or adhesives. For example, the device may conform with various concavities, convexities, ridges, depressions and/or lips, such that movement of the device is limited. Superior and/or inferior surfaces of the implant may include one or more concavities and/or one or more convexities,

The implants described herein can have varying curvatures and radii within the same plane, e.g. anteroposterior or mediolateral or superoinferior or oblique planes, or within multiple planes. In this manner, the articular surface repair system can be shaped to achieve an anatomic or near anatomic alignment between the implant and the implant site. This design not only allows for different degrees of convexity or concavity, but also for concave portions within a predominantly convex shape or vice versa. The surface of the implant that mates with the joint being repaired can have a variable geography that can be a function of the physical damage to the joint surface being repaired. Although, persons of skill in the art will recognize that implants can be crafted based on typical damage patterns, implants can also be crafted based on the expected normal congruity of the articular structures before the damage has occurred.

Moreover, implants can be crafted accounting for changes in shape of the opposing surfaces during joint motion. Thus, the implant can account for changes in shape of one or more articular surface during flexion, extension, abduction, adduction, rotation, translation, gliding and combinations thereof.

The devices described herein may be marginally translatable and self-centering. Thus, during natural articulation of a joint, the device is allowed to move slightly, or change its position as appropriate to accommodate the natural movement of the joint. The device does not, however, float freely in the joint. Further, upon translation from a first position to a second position during movement of a joint, the device tends to returns to substantially its original position as the movement of the joint is reversed and the prior position is reached. As a result, the device tends not to progressively “creep” toward one side of the compartment in which it is located. The variable geography of the surface along with the somewhat asymmetrical shape of the implant facilitates the self-centering behavior of the implant.

The device can also remain stationary over one of the articular surface. For example, in a knee joint, the device can remain centered over the tibia while the femoral condyle is moving freely on the device. The somewhat asymmetrical shape of the implant closely matched to the underlying articular surface helps to achieve this kind of stabilization over one articular surface.

For example, the implant shape may incorporate the shape of the joint on which it is positioned, such as portions of the tibial spines. Adding conformity with the tibial spines, e.g. the base of the tibial spines, can help in stabilizing the implant relative to the tibial plateau.

The motion within the joint of the devices described herein can optionally, if desired, be limited by attachment mechanisms. These mechanisms can, for example, allow the device to rotate, but not to translate. It can also allow the device to translate in one direction, while preventing the device from translating into another direction. The mechanisms can furthermore fix the devices within the joint while allowing the device to tilt. Suitable attachment mechanisms include ridges, pegs, pins, cross-members, teeth and protrusions. The configuration of these mechanisms can be parallel to one another, or non-parallel in orientation. The mechanisms can be pyramidal, triangular, spheroid, conical, or any shape that achieves the result. One or more attachment mechanism can be provided. Where more than one mechanism is provided, the mechanisms can cover the entire surface of the device, or a portion of the surface. Additional stabilization mechanisms can be provided such as ridges, lips and thickenings along all or a portion of a peripheral surface. For example, the stabilization mechanism may engage a peripheral edge of the tibial surface.

The implant height or profile selected can be chosen to alter the load bearing ability relative to the joint. Additionally the implant height can be adjusted to account for anatomic malalignment of bones or articular structures. Additionally, the implant taught herein in the presence of ligamentous laxity, the implant height, profile or other dimension can be adjusted to allow tightening of the ligament apparatus to improve the function. This occurs preferably without substantially interfering with axis alignment of the bones. Typically, the joints of are able to withstand up to 100% of the shear force exerted on the joint in motion.

Turning now to an illustrative example of an interpositional joint implant for implantation in a knee joint according to the scope and teachings of the invention. It is to be understood that an interpositional implant of the present invention may be applied to a wide variety of joints, including, without limitation, a hip joint, an ankle joint, a toe joint, a shoulder joint, an elbow joint, a wrist joint, and a finger joint.

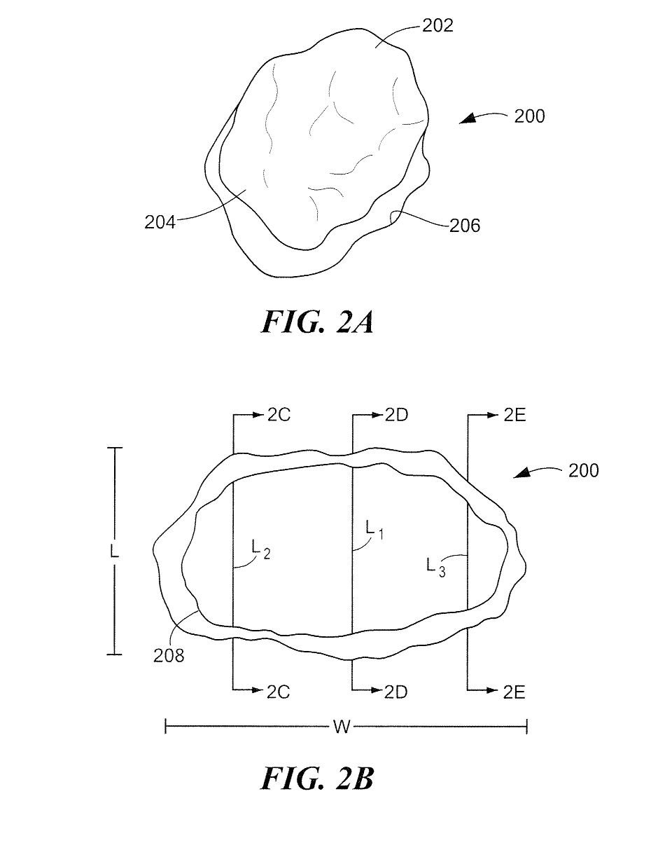

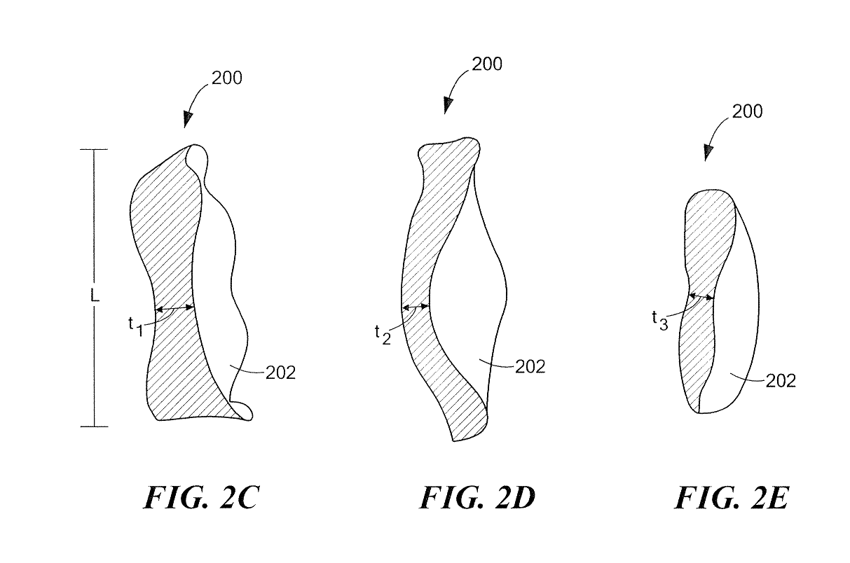

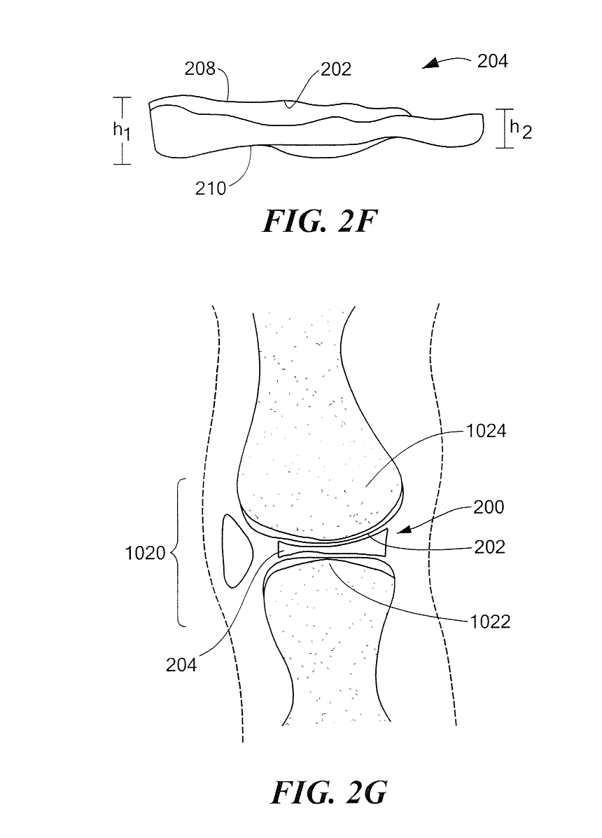

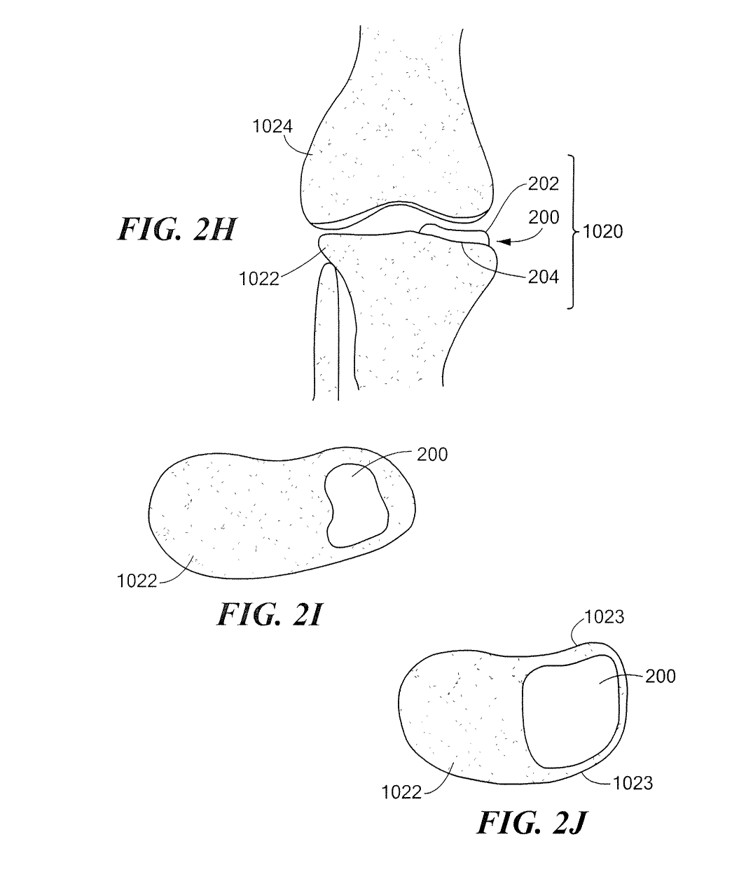

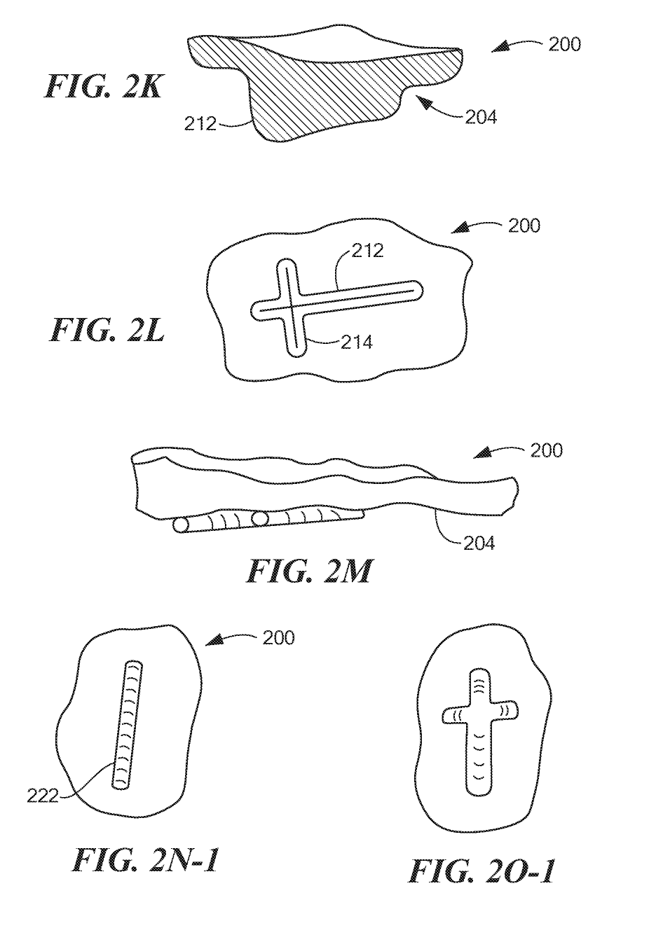

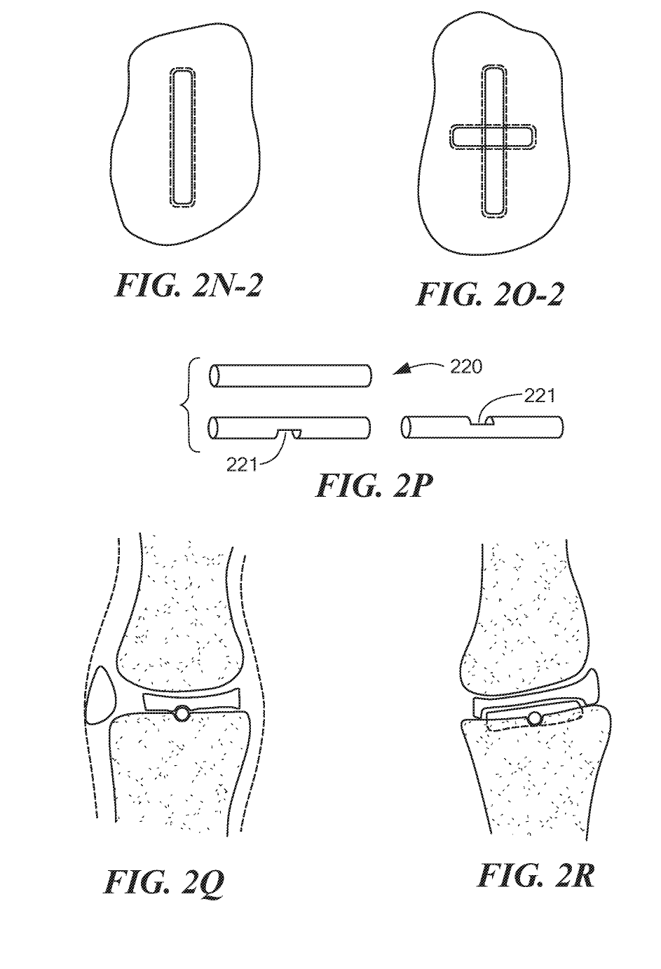

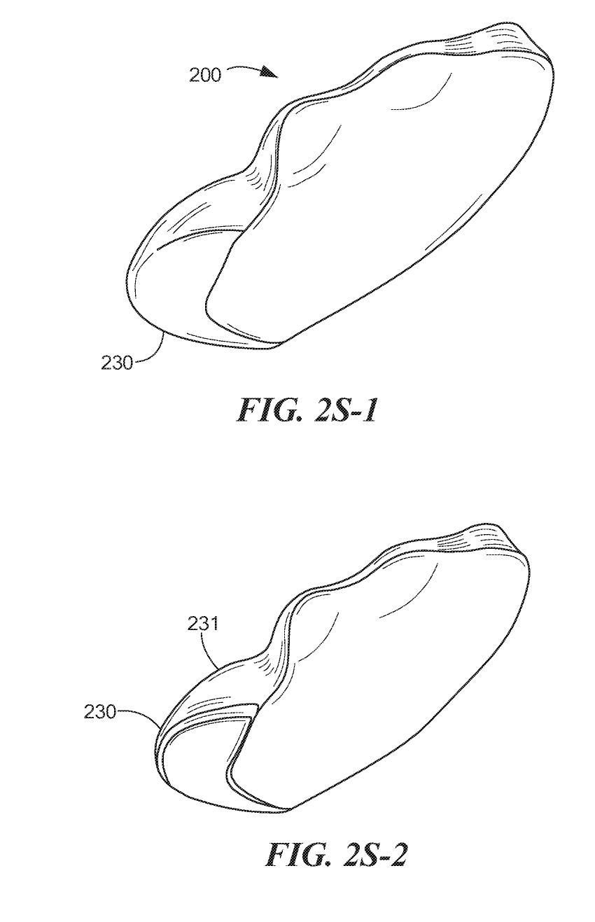

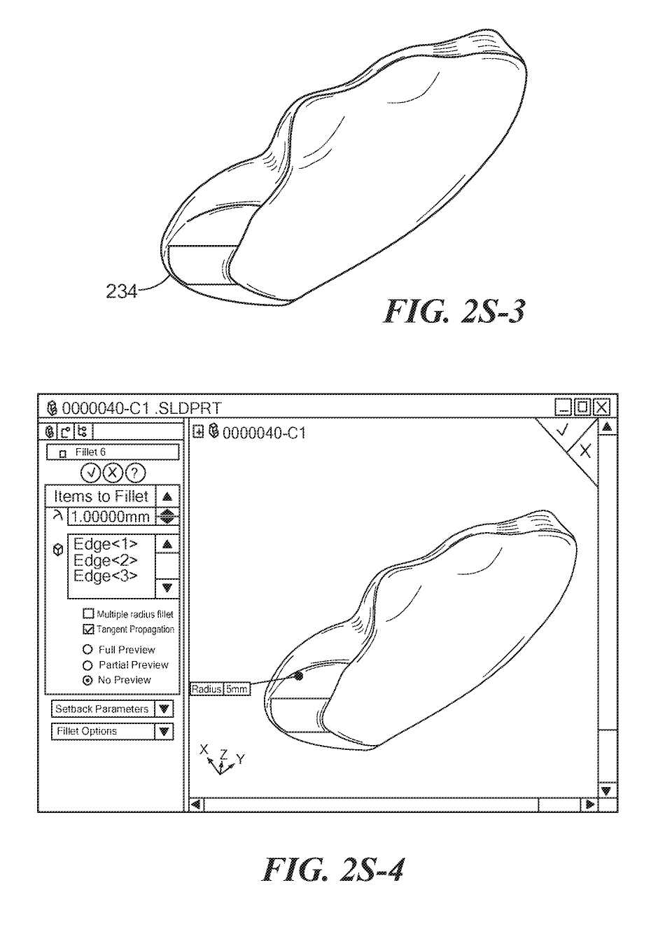

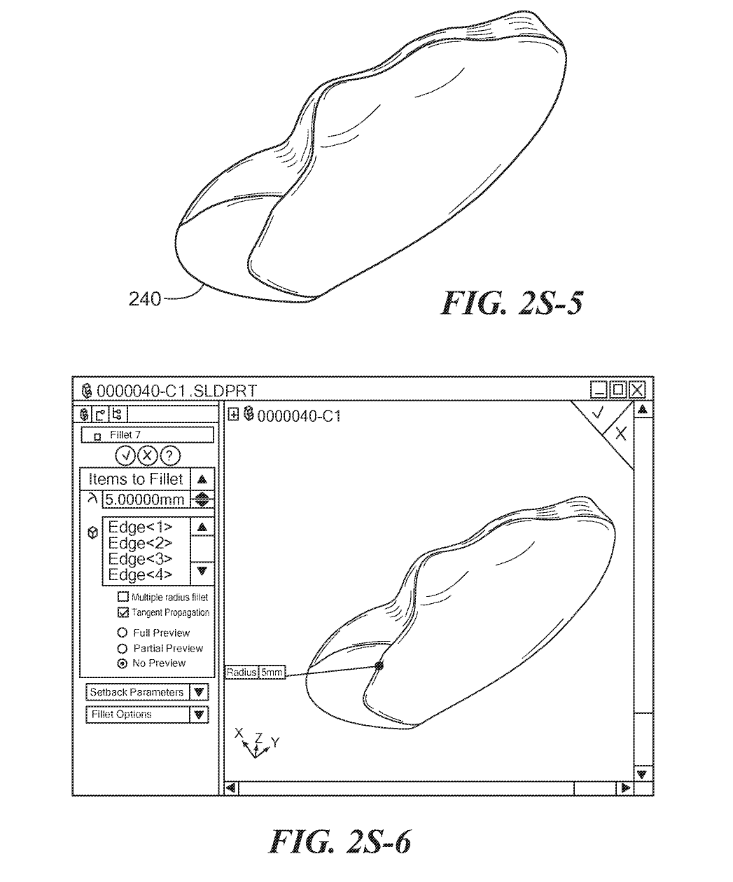

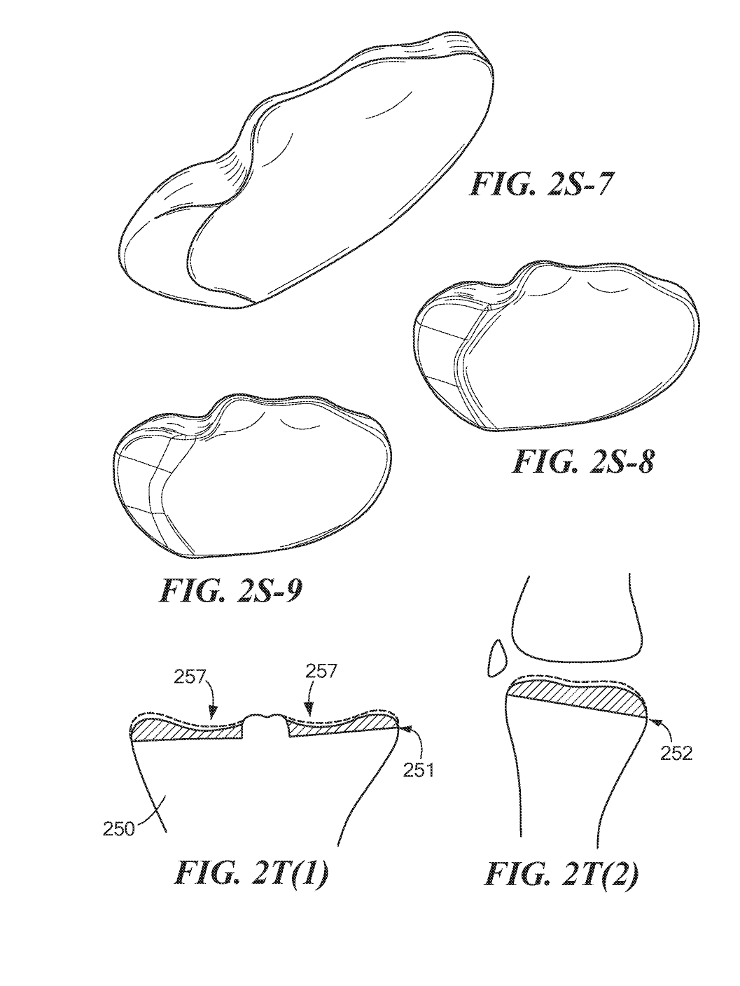

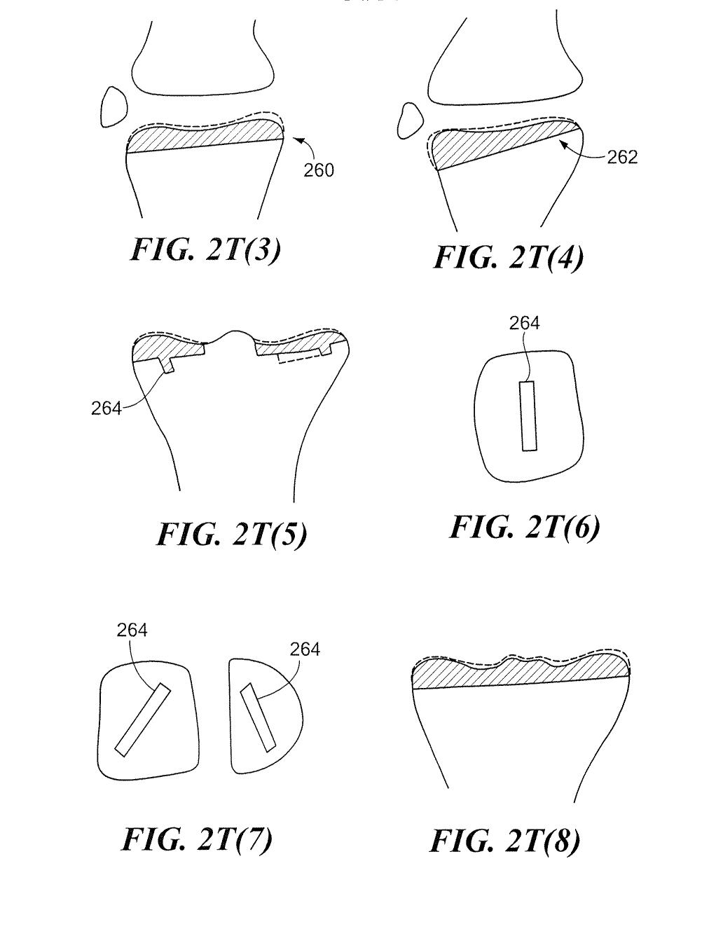

FIG. 2A shows a slightly perspective top view of a joint implant 200 of the invention suitable for implantation at the tibial plateau of the knee joint. As shown in FIG. 2A, the implant can be generated using, for example, a dual surface assessment, as described above with respect to FIGS. 1A and B.

The implant 200 has an upper surface 202, a lower surface 204 and a peripheral edge 206. The upper surface 202 is formed so that it forms a mating surface for receiving the opposing joint surface; in this instance partially concave to receive the femur. The concave surface can be variably concave such that it presents a surface to the opposing joint surface, e.g. a negative surface of the mating surface of the femur it communicates with. As will be appreciated by those of skill in the art, the negative impression, need not be a perfect one.

The upper surface 202 of the implant 200 can be shaped by any of a variety of means. For example, the upper surface 202 can be shaped by projecting the surface from the existing cartilage and/or bone surfaces on the tibial plateau, or it can be shaped to mirror the femoral condyle in order to optimize the complimentary surface of the implant when it engages the femoral condyle. In various embodiments, the upper surface 202 is substantially smooth in areas adapted to engage the femoral condyle, so as to permit movement of the condyle. More particularly, the upper surface 202 may be substantially free of irregularities, roughness, and projections in areas which are adapted to contact the femoral condyle. The upper surface 202 may be, without limitation, substantially concave, convex or flat. The upper surface 202 may include any combination of concavities and convexities. For example, the upper surface 202 may include, without limitation: a single concavity and at least one convexity; or a plurality of concavities and at least one convexity. The upper surface may have a substantially C-shape or U-shape cross-section in at least one of a medial-lateral direction and an anterior-posterior direction. In alternative embodiments, the superior surface 202 can be configured to mate with an inferior surface of an implant configured for the opposing femoral condyle.

The lower surface 204 typically has a convex surface that matches, or nearly matches, the tibial plateau of the joint such that it creates an anatomic or near anatomic fit with the tibial plateau. In various embodiments, the lower surface 204 may conform with: only cartilage of the tibial plateau; both cartilage and bone of the tibial plateau; or only bone of the tibial plateau. Thus, the lower surface 204 presents a surface to the tibial plateau that fits within the existing surface. It can be formed to match the existing surface (in embodiments, for example, that do not require making surgical cuts on the tibial surface) or to match the surface after articular resurfacing.

The lower surface 204 substantially conforming with the surface of the tibial plateau advantageously may limit movement of the implant in the joint. The lower surface 204 may be adapted to substantially remain fixed to the tibial plateau upon a load being placed on the upper surface 202. In various embodiments, the movement of the implant in the joint is thus limited without the use of pin, anchors and/or adhesives. As described above, the lower surface 204 may conform with a portion of the tibial spine area so as to limit movement of the implant.

As will be appreciated by those of skill in the art, the convex surface of the lower surface 204 need not be perfectly convex. Rather, the lower surface 204 is more likely consist of convex and concave portions that fit within the existing surface of the tibial plateau or the re-surfaced plateau. Thus, the surface is essentially variably convex and concave. The lower surface 204 may include any combination of concavities and convexities. For example, the lower surface 204 may include, without limitation: a single convexity and at least one concavity; or a plurality of convexities and at least one concavity. In various embodiments, the lower surface 204 may have a substantially inverted C-shape or U-shape cross-section in at least one of a medial-lateral direction and an anterior-posterior direction.

FIG. 2B shows a top view of the joint implant of FIG. 2A. As shown in FIG. 2B the exterior shape 208 of the implant can be elongated. The elongated form can take a variety of shapes including elliptical, quasi-elliptical, race-track, etc. However, as will be appreciated the exterior dimension is typically irregular thus not forming a true geometric shape, e.g. ellipse. As will be appreciated by those of skill in the art, the actual exterior shape of an implant can vary depending on the nature of the joint defect to be corrected. Thus the ratio of the length L to the width W can vary from, for example, between 0.25 to 2.0, and more specifically from 0.5 to 1.5. As further shown in FIG. 2B, the length across an axis of the implant 200 varies when taken at points along the width of the implant. For example, as shown in FIG. 2B, L₁≠L₂≠L₃.

Turning now to FIGS. 2C-E, cross-sections of the implant shown in FIG. 2B are depicted along the lines of C-C, D-D, and E-E. The implant has a thickness t1, t2 and t3 respectively. As illustrated by the cross-sections, the thickness of the implant varies along both its length L and width W The actual thickness at a particular location of the implant 200 is a function of the thickness of the cartilage and/or bone to be replaced and the joint mating surface to be replicated. In various embodiments, the implant has a peripheral edge with a greatest thickness that is at least 2 to 7 mm more than the smallest thickness within the implant. Further, the profile of the implant 200 at any location along its length L or width W is a function of the cartilage and/or bone to be replaced.

FIG. 2F is a lateral view of the implant 200 of FIG. 2A. In this instance, the height of the implant 200 at a first end h₁ is different than the height of the implant at a second end h₂. Further the upper edge 208 can have an overall slope in a downward direction. However, as illustrated the actual slope of the upper edge 208 varies along its length and can, in some instances, be a positive slope. Further the lower edge 210 can have an overall slope in a downward direction. As illustrated the actual slope of the lower edge 210 varies along its length and can, in some instances, be a positive slope. As will be appreciated by those of skill in the art, depending on the anatomy of an individual patient, an implant can be created wherein h₁ and h₂ are equivalent, or substantially equivalent without departing from the scope of the invention. In various embodiments, the peripheral edge of the implant may have a greatest height (relative to the lower surface 204 at its lowest point), that is larger than the smallest height of the upper surface 202 (relative to the lower surface 204 at its lowest point), within the implant by a ratio of 2:1, 3:1, 4:1 or 5:1. In still other embodiments, the lowest point of the central portion of the upper surface 202 may be lower than 30%, 40% or 50% of the perimeter defined by the varying center of the peripheral edge.

FIG. 2G is a cross-section taken along a sagittal plane in a body showing the implant 200 implanted within a knee joint 1020 such that the lower surface 204 of the implant 200 lies on the tibial plateau 1022 and the femur 1024 rests on the upper surface 202 of the implant 200. FIG. 2H is a cross-section taken along a coronal plane in a body showing the implant 200 implanted within a knee joint 1020. As is apparent from this view, the implant 200 is positioned so that it fits within a superior articular surface 224. As will be appreciated by those of skill in the art, the articular surface could be the medial or lateral facet, as needed.

FIG. 2I is a view along an axial plane of the body showing the implant 200 implanted within a knee joint 1020 showing the view taken from an aerial, or upper, view. FIG. 2J is a view of an alternate embodiment where the implant is a bit larger such that it extends closer to the bone medially, i.e. towards the edge 1023 of the tibial plateau, as well as extending anteriorly and posteriorly.

FIG. 2K is a cross-section of an implant 200 of the invention according to an alternate embodiment. In this embodiment, the lower surface 204 further includes a protrusion that serves as a joint anchor 212. As illustrated in this embodiment, the joint anchor 212 forms a keel or vertical member that extends from the lower surface 204 of the implant 200 and projects into, for example, the bone of the joint. As will be appreciated by those of skill in the art, the keel can be perpendicular or lie within a plane of the body. The joint anchor 212 may be inserted, for example, into a cut made in the tibial plateau, such that motion of the implant is substantially limited.

As shown in FIG. 2K, the joint anchor 212 may include a taper. The addition of the taper in, without limitation, an anterior to posterior direction on the lowest surface of the joint anchor 212, can allow for easier insertion of the implant into the joint.

Additionally, as shown in FIG. 2L the joint anchor 212 can have a cross-member 214 so that from a bottom perspective, the joint anchor 212 has the appearance of a cross or an “x.” As will be appreciated by those of skill in the art, the joint anchor 212 could take on a variety of other forms while still accomplishing the same objective of providing increased stability of the implant 200 in the joint. These forms include, but are not limited to, pins, bulbs, balls, teeth, etc. Additionally, one or more joint anchors 212 can be provided as desired. The joint anchors 212 may be positioned to be symmetrical, asymmetrical, rows, and random.

FIGS. 2M and N illustrate cross-sections of alternate embodiments of a dual component implant from a side view and a front view. In the alternate embodiment shown in FIG. 2M it may be desirable to provide a one or more cross-members 220 on the lower surface 204 in order to provide a bit of translation movement of the implant relative to the surface of the femur, or femur implant. In that event, the cross-member can be formed integral to the surface of the implant or can be one or more separate pieces that fit within a groove 222 on the lower surface 204 of the implant 200. The groove can form a single channel as shown in FIG. 2N-1, or can have more than one channel as shown in FIG. 2O-1. In either event, the cross-bar then fits within the channel as shown in FIGS. 2N-2 and 2O-2. The cross-bar members 220 can form a solid or hollow tube or pipe structure as shown in FIG. 2P. Where two, or more, tubes 220 communicate to provide translation, a groove 221 can be provided along the surface of one or both cross-members to interlock the tubes into a cross-bar member further stabilizing the motion of the cross-bar relative to the implant 200. As will be appreciated by those of skill in the art, the cross-bar member 220 can be formed integrally with the implant without departing from the scope of the invention.

As shown in FIGS. 2Q-R, it is anticipated that the surface of the tibial plateau will be prepared by forming channels thereon to receive the cross-bar members. Thus facilitating the ability of the implant to seat securely within the joint while still providing movement about an axis when the knee joint is in motion.

FIG. 2S(1-9) illustrate an alternate embodiment of implant 200. As illustrated in FIG. 2S the edges are beveled to relax a sharp corner. FIG. 2S(1) illustrates an implant having a single fillet or bevel 230. The fillet is placed on the implant anterior to the posterior portion of the tibial spine. As shown in FIG. 2S(2) two fillets 230, 231 are provided and used for the posterior chamfer. In FIG. 2S(3) a third fillet 234 is provided to create two cut surfaces for the posterior chamfer.

Turning now to FIG. 2S(4) a tangent of the implant is deselected, leaving three posterior curves. FIG. 2S(5) shows the result of tangent propagation. FIG. 2S(6) illustrates the effect on the design when the bottom curve is selected without tangent propagation. The result of tangent propagation and selection is shown in FIG. 2S(7). As can be seen in FIG. 2S(8-9) the resulting corner has a softer edge but sacrifices less than 0.5 mm of joint space. As will be appreciated by those of skill in the art, additional cutting planes can be added without departing from the scope of the invention.

FIG. 2T illustrates an alternate embodiment of an implant 200 wherein the surface of the tibial plateau 250 is altered to accommodate the implant. As illustrated in FIG. 2T(1-2) the tibial plateau can be altered for only half of the joint surface 251 or for the full surface 252. As illustrate in FIG. 2T(3-4) the posterior-anterior surface can be flat 260 or graded 262. Grading can be either positive or negative relative to the anterior surface. Grading can also be used with respect to the implants of FIG. 2T where the grading either lies within a plane or a body or is angled relative to a plane of the body. Additionally, attachment mechanisms can be provided to anchor the implant to the altered surface. As shown in FIG. 2T(5-7) keels 264 can be provided. The keels 264 can either sit within a plane, e.g. sagittal or coronal plane, or not sit within a plane (as shown in FIG. 2T(7)). FIG. 2T(8) illustrates an implant which covers the entire tibial plateau. The upper surface of these implants are designed to conform to the projected shape of the joint as determined under the steps described with respect to FIG. 1, while the lower surface is designed to be flat, or substantially flat to correspond to the modified surface of the joint.

The foregoing description of embodiments of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention and the various embodiments and with various modifications that are suited to the particular use contemplated.

What is claimed is:

1. An articular implant for replacing or repair a diseased or damaged joint of a patient, a first implant surface and a periphery, wherein the first implant surface is configured to conform a first articular surface of the diseased or damaged joint of the patient based on electronic image data of the diseased and damaged joint of the patient, wherein the periphery includes a stabilization mechanism configured to limit motion of the articular implant upon implantation.

2. The articular implant of claim 1, wherein the first articular surface of the diseased or damaged joint of the patient includes cartilage, or subchondral bone, or both.

3. The articular implant of claim 1, wherein the stabilization mechanism includes a ridge or a lip.

4. The articular implant of claim 1, wherein the stabilization mechanism is located along a portion of the periphery.

5. The articular implant of claim 1, wherein the diseased or damaged joint of a patient is a knee joint, and wherein the first articular surface is a condylar surface of the knee joint.
